(19) United States
(12) Patent Application Publication
(10) Pub. No.: US 2021/0198663 A1
(43) Pub. Date: Jul. 1, 2021

(54) Nanowires/Microscale Pyramids (NWs/MPs) Complex Structure, Method for manufacturing the Same and Its Applications to Isolation of Circulating tumor cells (CTCs) and Detection of Epstein-Barr virus (EBV) DNA

(71) Applicants: Sheng Wei Lee, Taipei City (TW); Ming Ying Lan, Taipei City (TW)

(72) Inventors: Sheng Wei Lee, Taipei City (TW); Ming Ying Lan, Taipei City (TW)

(21) Appl. No.: 16/726,949

(22) Filed: Dec. 26, 2019

(51) Int. Cl. C12N 15/11; C12Q 1/70

(52) U.S. Cl. CPC C12N 15/11; C12N 2310/351; C12Q 1/705

(57) ABSTRACT

A nanowires/microscale pyramids (NWs/MPs) substrate complex structure is formed with a plurality of pyramids. Each triangular surface of the pyramid is formed with nanowires to increase the contact area. In one application, anti-epithelial-cell adhesion-molecule (anti-EpCAM) antibodies are modified on the nanowires. The anti-EpCAM antibodies serve to capture circulating tumor cells (CTCs) in blood for determining whether the cancer cells have metastasized to other organs. In another application, the nanowires are modified with silver nanoparticles (AgNPs) which can be combined with other functional groups for testing. The AgNPs on the NWs/MPs substrate causes the NWs/MPs substrate to become a substrate with surface enhanced Raman scattering (SERS). The AgNPs are bound with Epstein-Barr virus (EBV) probe DNA which can be hybridized with EBV target DNAs so as to determine the concentration of the EBV target DNAs in blood. The methods for fabricating the substrate and its applications are also provided.

### FIELD OF THE INVENTION

The present invention is related to nanowires/microscale pyramids (NWs/MPs) substrates, and in particular to a NWs/MPs substrate complex structure, an application about detections of circulating tumor cells (CTC) and Epstein-Barr virus (EBV) target DNAs and a method for fabricating the NWs/MPs substrate complex structure.

### BACKGROUND OF THE INVENTION

The prior art has proposed a method for fabrication of nanowires on a silicon substrate which serves to detect bio-chemical material in blood. In the prior art, the silicon substrate is formed with a plurality of nanowires. Then some chemical materials are added on the nanowires. The chemical material serves to combine with specific components in blood so as to determine whether these components are contained in blood. This technology is mainly used in detection of cancers.

In cancers research, detection of Circulating tumor cells (CTC) is a valuable method for determining whether cancer cells have metastasized to other organs. The metastasis of cancer cells is a primary reason to cause the cancer patients to die. Therefore, the detection of CTCs is a useful tool to determine whether the cancer cells have metastasized to other organs of the human body. In fact, many chemical or physical methods have been developed for detecting CTCs, but most are without high sensitivity. The presence of CTCs not only reveals active metastasis, but also helps to monitor disease progression by providing real-time information about the patient's disease status. Moreover, it helps to facilitate and determine appropriate tailored treatments for patients.

Plasma levels of Epstein-Barr virus (EBV) DNA has been found to be highly correlated to the disease status of the Nasopharyngeal carcinoma (NPC) patients. It is typically measured by real-time quantitative polymerase chain reaction (PCR) which is usually time-consuming. Until now, there is still no definite examination to determine whether a nasopharyngeal carcinoma patient has been totally discovered from disease.

Therefore, the object of the present invention is to provide a silicon NWs/MPs substrate complex structure for detection of CTC and EBV target DNAs by using the complex structure and the methods for fabricating them so as to resolve the above mentioned prior art problems.

### SUMMARY OF THE INVENTION

Accordingly, for improving above mentioned drawbacks in the prior art, the object of the present invention is to provide a NWs/MPs substrate complex structure, an application about detections of CTC and EBV target DNAs and a method for fabricating the NWs/MPs substrate complex structure, wherein the NWs/MPs substrate is formed with a plurality of pyramids 20 thereon. Each triangular surface of the pyramid is formed with nanowires so as to increase the contact area with a detected or tested object and to have a preferred detection effect. Furthermore, the present invention proposes two applications. In first application, anti-epithelial-cell adhesion-molecule (anti-EpCAM) antibodies are modified on the nanowires 30. The anti-EpCAM antibodies serve to capture CTC in blood. This application is used to determine whether the cancer cells have metastasized to other organs. In the second application, the nanowires are modified with silver nanopartilces (AgNPs) which can be combined with other functional groups for testing. The AgNPs on the NWs/MPs substrate will cause the NWs/MPs substrate to become a substrate with surface enhanced Raman scattering (SERS). In the present invention the AgNPs are bound with EBV probe DNA which has the function of hybridizing with EBV target DNAs so as to determine concentration of the EBV target DNAs in blood. The NWs/MPs substrate can also be used to detect CTC of other cancers. The nanowires 30 may be modified with different kinds of antibodies, such as anti-EpCAM antibody, anti-vimentin, anti-cytokeratin 8/18, or anti-CD44. Using various kinds of antibodies on the nanowire can increase the probabilities of detections of diseases.

To achieve above object, the present invention provides a NWs/MPs substrate complex structure, comprising: a NWs/MPs substrate; an upper surface of the NWs/MPs substrate being formed as a plurality of pyramids; each pyramid having an approximate rectangular bottom; an upper side of each pyramid being formed by four triangular surfaces; the bottom of the pyramid having a width between 5 μm to 100 μm and a height between 10 μm to 100 μm; each triangular surface of the pyramid being formed with a plurality of nanowires which has length between 0.3 μm to 30 μm; wherein NWs/MPs substrate serves to carry chemical elements, the chemical elements are carried on the nanowires 30 for bio-test, especially, for blood tests.

Furthermore, the method for fabricating NWs/MPs substrate complex structure comprises steps of: ultrasonically cleaning a (100) oriented Si wafer (Boron-doped 1-10 Ω·cm) in acetone, isopropanol, and ethanol to remove contaminants; etching cleaned Si wafer in solution containing potassium hydroxide (KOH), and isopropanol at temperature greater than 80° C. for a predetermined time to produce micrometric pyramids on the Si substrate so as to form a Si pyramid substrate; dipping the Si pyramid substrate into solution of hydrofluoric acid (HF) and 0.1 N silver nitrate (AgNO3) at 25° C.; depositing Ag nanoclusters on surfaces of Si pyramid substrate; etching the Si pyramid substrate in a solution of HF and Fe(NO3)3.9H2O to produce the so-called Si NWs/MPs substrate with different wire lengths; and removing residual Ag nanoclusters on the surface by the ultrasonic vibration; wherein in above process, volumes and weights of all components in the process can be increased or decreased with the same ratio; this variation will not affect the fabricating process; and furthermore each value has a variation of ±20%.

### BRIEF DESCRIPTION OF THE DRAWINGS

FIG. 1 is a schematic illustration showing the silicon NWs/MPs substrate capturing CTCs.

FIG. 2A is a SEM view of different silicon NWs/MPs substrates, wherein lengths of nanowires are 1.5 μm-3.8 μm-5.1 μm and 6.9 μm.

FIG. 2B shows fabricating steps of coating AgNPs on the silicon NWs/MPs substrate.

FIG. 3 shows that the cell capture efficiency for different silicon NWs/MPs substrates which are generated from the second etching. It is shown that the etching time of 20 minutes has better effect.

FIG. 4 is an SEM image showing the capturing of CTCs by silicon NWs/MPs substrate.

FIG. 5 is a schematic illustration of EBV DNA captured on AgNPs-coated NWs/MPs and detected by Raman spectroscope.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

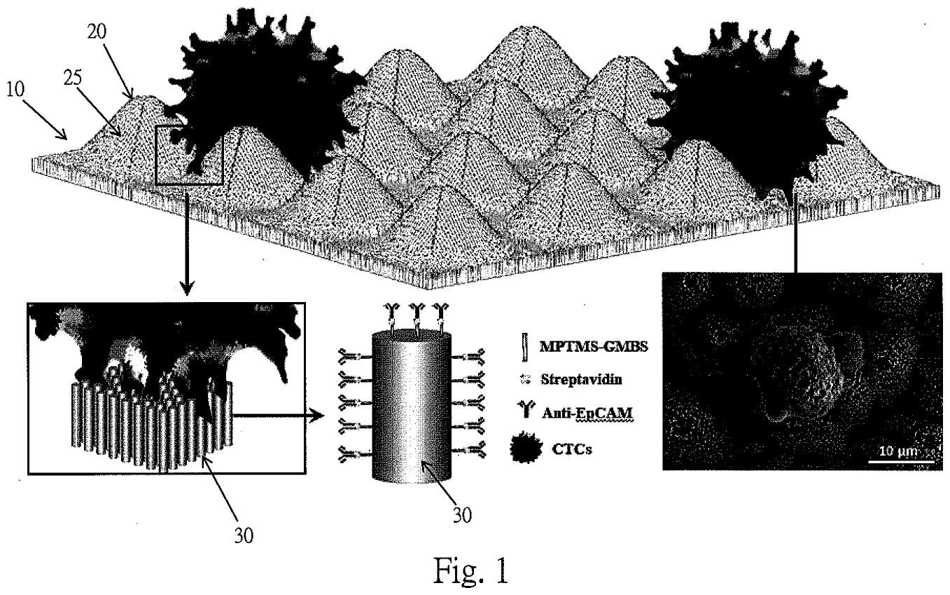

With reference to FIG. 1, the NWs/MPs substrate complex structure of the present invention is illustrated. The present invention includes the following elements.

A NWs/MPs substrate (nanowires/microscale pyramids (NWs/MPs) structures) 10 is provided. The upper surface of the NWs/MPs substrate is formed as a plurality of pyramids 20. As illustrated in FIG. 1, each pyramid 20 has an approximate rectangular bottom. An upper side of each is pyramid 20 is formed by four triangular surfaces 25. The bottom of the pyramid 20 has a width between 5 μm to 100 μm and a height between 10 μm to 100 μm.

Each triangular surface 25 of the pyramid 20 is formed with a plurality of nanowires 30 which has length between 0.3 μm to 30.0 μm.

The material of NWs/MPs substrate can be selected from carbon, silicon or germanium.

In the present invention, the NWs/MPs substrate serves to carry chemical elements, especially, the chemical elements are carried on the nanowires 30 for bio-test, especially, for blood tests.

In the present invention, the NWs/MPs substrate has two applications.

The first application of the present invention is to modify it with anti-epithelial-cell adhesion-molecule (anti-EpCAM) antibodies. As illustrated in FIG. 1, the surface of the Si NWs/MPs substrate is first modified with 3-mercaptopropyl trimethoxysilane (MPTMS), then, N-maleimidobutyryloxy succinimide ester (GMBS) is added as a coupling agent, followed by streptavidin (SA). Finally, the modified substrate is conjugated with anti-EpCAM antibodies. The anti-EpCAM antibodies have the function of capturing Circulating tumor cells (CTCs) in blood. CTCs, are cancer cells shed from the primary tumor which circulate in the bloodstream, creating opportunities for metastatic spread. Therefore, by this way, early metastasis can be determined. In the present invention, the nanowires 30 may be modified with different kinds of antibodies, such as anti-EpCAM antibody, anti-vimentin, anti-cytokeratin 8/18, or anti-CD44. Using various kinds of antibodies on the nanowire 30 can increase the probabilities of detections of CTCs.

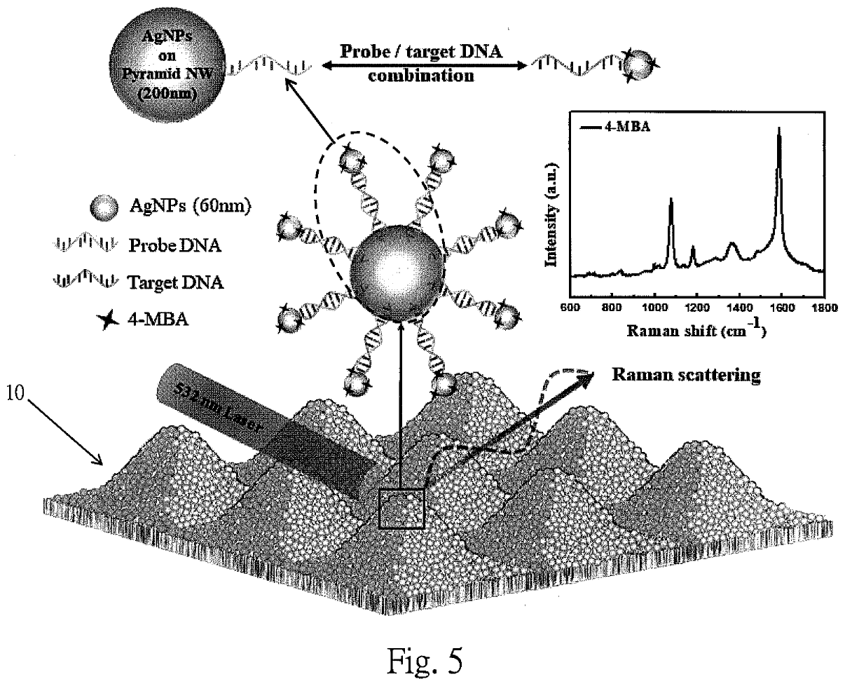

In the second application, the nanowire 30 is coated with Ag nanoparticles (AgNPs), therefore the NWs/MPs substrate has the effect of surface enhanced Raman scattering (SERS). The AgNPs may be further bound with Epstein-Barr virus (EBV) probe DNA. The EBV probe DNA has the function of hybridizing EBV target DNA, as illustrated in FIG. 5, a Raman scattering spectroscope is used to measure the concentration of EBV target DNAs in blood.

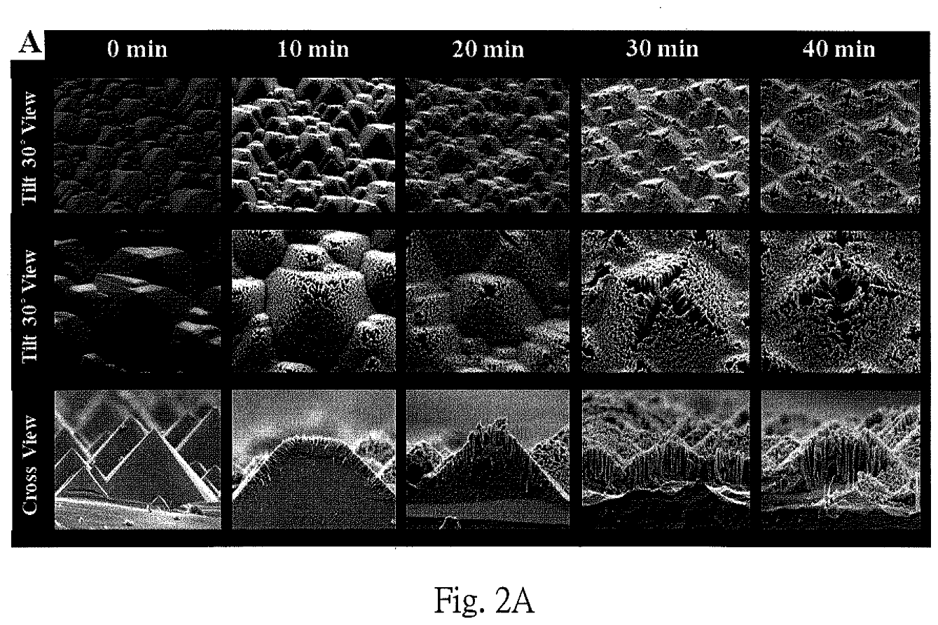

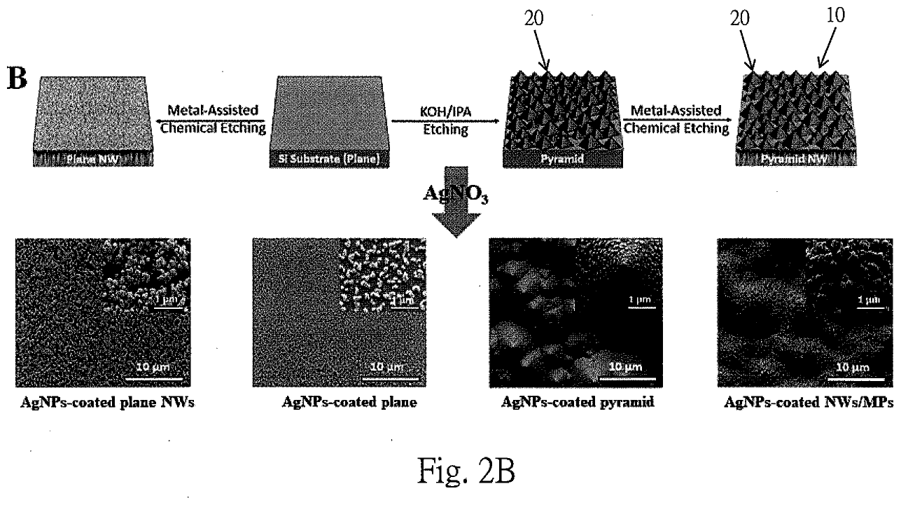

(A) In the following, a way for fabricating NWs/MPs substrate compound structure according to the present invention is illustrated. With reference to FIG. 2, the process contains the steps of:

Ultrasonically cleaning a (100) oriented Si wafer (Boron-doped 1-10 Ω·cm) in acetone, isopropanol, and ethanol each for 5 minutes to remove contaminants.

The cleaned Si wafer is then etched (a first etching step) in solution containing 600 ml deionized water (DI-water), 55 ml potassium hydroxide (KOH), and 45 ml isopropanol for 1 h at 82° C. to produce micrometric pyramids on the Si substrate so as to form a Si pyramid substrate. The material of NWs/MPs substrate can be selected from carbon, silicon, or germanium.

Then, dipping the Si pyramid substrate into the solution of 78 ml DI-water, 22 ml hydrofluoric acid (HF), and 2 ml 0.1 N silver nitrate (AgNO3) at 25° C. through 30 seconds, depositing Ag nanoclusters on the surface of pyramid substrate.

Etching (a second etching step) the pyramid substrate in a solution of 78 ml DI-water, 22 ml HF, and 6.8 g Fe(NO3)3.9H2O for 10, 20, 30, 40 minutes to produce the so-called Si NWs/MPs substrate with different wire lengths; and then removing residual Ag nanoclusters on the surface by the ultrasonic vibration.

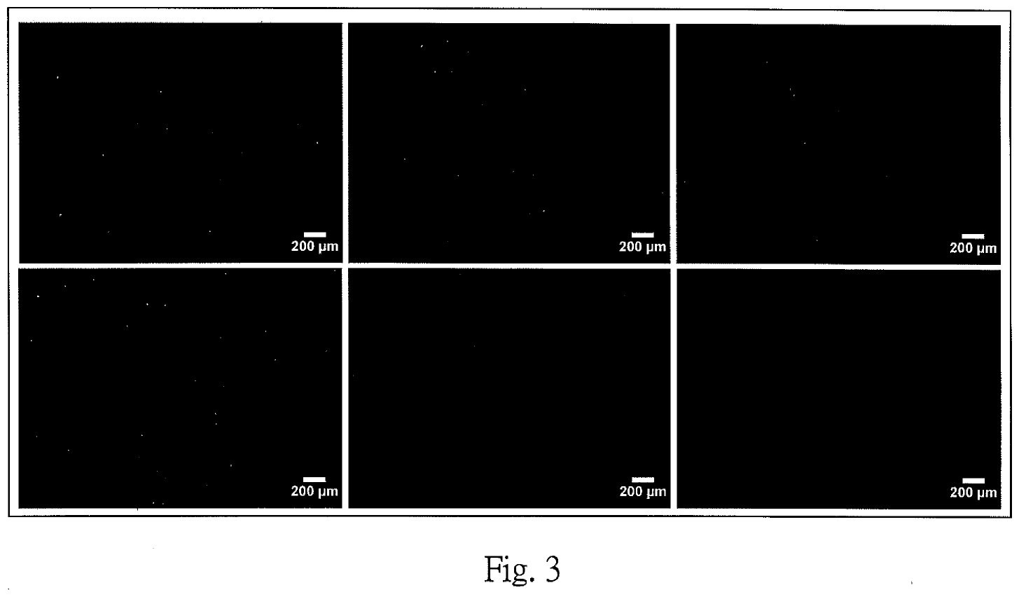

For the etching time of 10 to 40 minutes, the nanowires 30 derived have lengths of 1.5 μm to 6.9 μm. In this example, the etching time 10, 20, 30, 40 minutes, and the nanowires 30 derived have lengths of 1.5 μm, 3.8 μm, 5.1 μm and 6.9 μm, respectively, as shown in FIG. 2A. FIG. 3 shows the cell capture efficiency for different NWs/MPs substrates which are generated from the second etching. It is shown that the etching time of 20 minutes has better effect. At this etching time period, the nanowire 30 acquired is not the longest one, but heights of the pyramids 20 can be retained with an optimum one.

In above process, the volumes and weights of all components in the process can be increased or decreased with the same ratio. This variation will not affect the fabricating process of the present invention. Furthermore, each value may have a variation of ±20%.

(B) The way for forming AgNPs on the nanowire 30 of the NWs/MPs substrate will be described herein.

For EBV DNA detection, the Si NWs/MPs substrates are further dipped in the 0.002M AgNO3 for 1 minute to fabricate the AgNPs-coated NWs/MPs so that the NWs/MPs substrate has the function of surface enhancing Raman scattering (SERS). FIG. 2B shows a schematic view about the fabrication of this process. In above process, the volumes and weights of all components in the process can be increased or decreased with the same ratio. This variation will not affect the fabricating process of the present invention. Furthermore, each value may have a variation of ±20%.

In the first application of the present invention, the NWs/MPs substrate is modified with anti-EpCAM antibodies, the process for modification will be described herein.

The surface of the NWs/MPs substrate is first modified with 4% (v/v) 3-mercaptopropyl trimethoxysilane (MPTMS) in absolute ethanol by silane chemistry for 1 h.

Then, 0.25 mM N-maleimidobutyryloxy succinimide ester (GMBS) in DMSO solution is added as a coupling agent for 1 h, followed by 10 pg/ml of streptavidin (SA) for 30 min at 25° C. There are two reactive groups in the GMBS molecule, maleimide and N-hydroxysuccinimide, which are reactive towards the sulfhydryl group in MPTMS and amino group in SA, respectively.

After removing excess SA by using PBS (Phosphate buffered saline), the modified NWs/MPs substrate is dipped into biotinylated antinylated anti-EpCAM (2 μs/ml in PBS, goat anti-human EpCAM polylclonal antibody, R & D System, Inc., USA) for 30 min, the anti-EpCAM antibodies will connect with the SA on the nanowire 30.

In the first application of the present invention, the NWs/MPs substrate is modified with anti-EpCAM antibodies, the process for modification will be described herein.

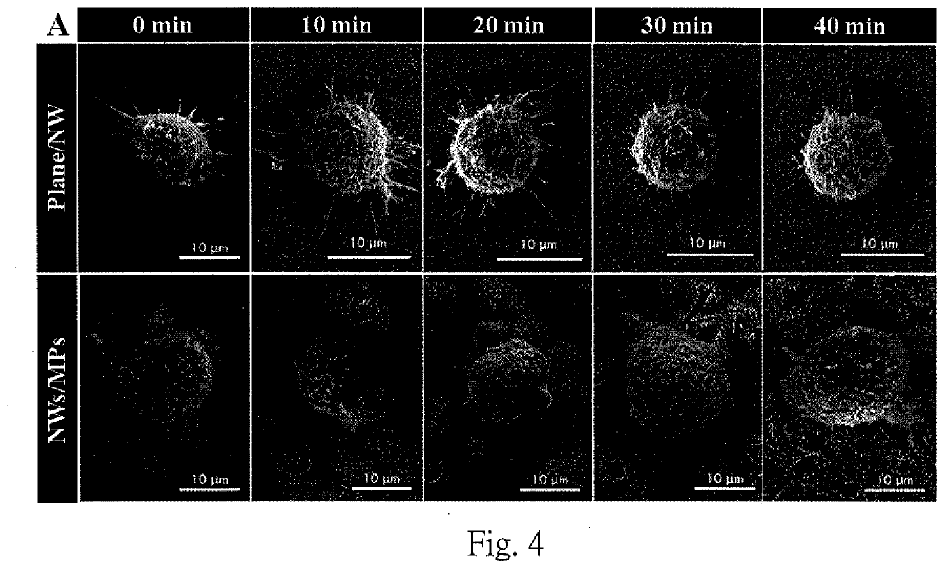

By above mentioned structure, the anti-EpCAM antibodies can capture the CTCs in the blood of a Nasopharyngeal carcinoma patient so as to determine the status of the disease of the Nasopharyngeal carcinoma patient. As a result, it is possible to early detect and monitor cancer progression and surveillance of therapeutic efficacy in NPC patients. FIGS. 1, 3 and 4 shows the effect of capturing of the CTCs.

In the second application of the present invention, the AgNPs on the NWs/MPs substrate is bound with the EBV probe DNAs. The EBV probe DNAs can hybridize with EBV target DNAs so as to determine the concentration of the EBV target DNAs in the blood. A Raman spectroscopy is used to determine whether the EBV target DNAs are captured. However, it is difficult for the Raman spectroscopy to detect the EBV target DNAs directly. It is necessary to combine the EBV target DNAs to another AgNPs which are different from the AgNPs coated on the nanowire 30. The AgNPs bound with the EBV target DNAs are further modified with 4-MBAs (4-Mercaptobenzoic acid) which can be detected by Raman spectroscopy easily.

In the followings, the method for modifying the 4-MBAs and EBV target DNAs to another AgNPs is described. 6 ul of DNA stock solution (1 uM) is added into 500 ul of 60 nm AgNPs (1 nM) and mixed by a brief vortexing to yield a final 500 ul EBV target DNA solution. 5 μl of 500 mM pH 3 citrate buffer is then added into the AgNPs solution and vortexed briefly, followed by incubation at room temperature for 5 minutes. The same amount of the pH 3 buffer solution is added into the DNA-AgNPs mixture again to achieve a final pH 3 10 mM buffer. After incubation at room temperature for 25 minutes for DNA loading, 15 μl of 500 mM pH 7.6 HEPES (4-(2-hydroxyethyl)-1-piperazineethanesulfonic acid) buffer is added to adjust the pH of the AgNP solution to neutral pH. The DNA-AgNPs mixture is then centrifuged for 30 minutes at 13000 rpm. The supernatant is removed, and the pellet is washed 2-3 times with 5 mM HEPES buffer, followed by centrifugation to remove free DNAs completely. The washed DNA-AgNPs is re-dispersed in 5 mM HEPES buffer (pH7.6) for further use.

Then taking 1 ml of aqueous dispersion of AgNPs modified with the EBV target DNA to mix with 0.5 ml of 4-MBA ethanol solution (0.1M) for 20 minutes. After 30 minutes of centrifugation (13000 rpm), the pellet comprising 4-MBA functionalized AgNPs is washed with ddH2O (distillation-distillation H2O) using centrifugation to get rid of unbound 4-MBA molecules. Finally, 4-MBA functionalized EBV target DNAs-AgNPs are re-dispersed in 1×PBS.

In above process, the volumes and weights of all components in the process can be increased or decreased with the same ratio. This variation will not affect the fabricating process of the present invention. Furthermore, each value may have a variation of ±20%.

In the following, the method about the hybridization of EBV target DNA with EBV probe DNA bound on the AgNPs-coated NWs/MPs substrate is described.

First, 0.5 ml of the EBV probe DNA (1 uM) is added on the AgNPs-coated NWs/MPs substrate and incubated for 2 hours at room temperature with shaking. Afterwards, they are washed 3 times with 1×PBS to remove unbound EBV probe DNAs, and

then treated with 0.5 ml silver nanoparticles modified with 4-MBA and EBV target DNA dispersing in 1×PBS for another 2 hours at room temperature with shaking.

Then the NWs/MPs substrates are thoroughly washed and immersed in ddH2O for 1 h.

Finally, the substrates are air-dried.

Then the SERS spectra of 4-MBA are collected using a Micro-Raman spectroscopic system (PTT-EL) with 532 nm excitation laser. The Raman spectra integration time is 20 sec for each location. Signals at 1078 and 1596 cm-1 indicate hybridization of target EBV DNA and probe DNA. FIG. 5 is a schematic illustration showing that EBV DNA captured on AgNPs-coated NWs/MPs and detected by Raman spectroscopy.

In above process, the volumes and weights of all components in the process can be increased or decreased with the same ratio. This variation will not affect the fabricating process of the present invention. Furthermore, each value may have a variation of ±20%.

Advantages of the present invention are that the NWs/MPs substrate is formed with a plurality of pyramids 20 thereon. Each triangular surface of the pyramid is formed with nanowires so as to increase the contact area with a detected or tested object so that the detection effect is preferred. Furthermore, the present invention proposes two applications. In the first application, anti-EpCAM antibodies are modified on the nanowires 30. The anti-EpCAM antibodies serve to catch CTCs in blood. This application is used to determine whether the cancer cells have metastasized to other organs. In the second application, the nanowires are modified with AgNPs which can be combined with other functional groups for testing. The AgNPs on the NWs/MPs substrate will cause the NWs/MPs substrate to become a substrate with surface enhanced Raman scattering (SERS). In the present invention the AgNPs are bound with EBV probe DNA which has the function of hybridize with EBV target DNAs so as to determine the concentration of the EBV target DNAs in blood. The NWs/MPs substrate can also be used to detect CTC of other cancers. The nanowires 30 may be modified with different kinds of antibodies, such as anti-EpCAM antibody, anti-vimentin, anti-cytokeratin 8/18, or anti-CD44. Using various kinds of antibodies on the nanowire 30 can increase the probabilities of detections of CTCs.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A NWs/MPs substrate complex structure, comprising:
a NWs/MPs substrate (nanowires/microscale pyramids (NWs/MPs) structures); an upper surface of the NWs/MPs substrate being formed as a plurality of pyramids; each pyramid having an approximate rectangular bottom; an upper side of each pyramid being formed by four triangular surfaces;
each triangular surface of the pyramid being formed with a plurality of nanowires;
wherein NWs/MPs substrate serves to carry chemical elements, the chemical elements are carried on the nanowires for bio-test, especially, using in blood tests.

2. The NWs/MPs substrate complex structure as claimed in claim 1, wherein the bottom of the pyramid having a width between 5 μm to 100 μm and a height between 10 μm to 100 μm.

3. The NWs/MPs substrate complex structure as claimed in claim 1, wherein the nanowires has length between 0.3 μm to 30.0 μm

4. The NWs/MPs substrate complex structure as claimed in claim 1, wherein material of the NWs/MPs substrate is selected from carbon, silicon, or germanium.

5. The NWs/MPs substrate complex structure as claimed in claim 1, wherein the nanowire is modified with one or several kinds of antibodies.

6. The NWs/MPs substrate complex structure as claimed in claim 1, wherein the nanowire is modified with at least one kind of antibodies selected from anti-EpCAM, anti-vimentin, anti-cytokeratin 8/18, and anti-CD44 antibodies.

7. The NWs/MPs substrate complex structure as claimed in claim 1 or claim 3, wherein 3-mercaptopropyl trimethoxysilane (MPTMS) are attached on the nanowire, then MPTMS is combined with N-maleimidobutyryloxy succinimide ester (GMBS), next the GMBS is combined with streptavidin (SA), and the SA is finally combined with antibodies.

8. The NWs/MPs substrate complex structure as claimed in claim 1, wherein the nanowire is coated with AgNPs (Ag nanoparticles) so that the substrate has the effect of surface enhanced Raman scattering (SERS).

9. The NWs/MPs substrate complex structure as claimed in claim 8, wherein the AgNPs are bound with EBV (Epstein-Barr virus) probe DNA for hybridization with EBV target DNAs so as to detect the concentration of EBV target DNAs in blood.

10. The method for fabricating NWs/MPs substrate complex structure comprising steps of:
ultrasonically cleaning a (100) oriented Si wafer (Boron-doped 1-10 Ω·cm) in acetone, isopropanol, and ethanol to remove contaminants;
etching cleaned wafer in solution containing potassium hydroxide (KOH) and isopropanol at temperature greater than 80° C. for a predetermined time to produce micrometric pyramids on the substrate so as to form a pyramid substrate;
then forming nanowires on the pyramid substrate containing the following steps of:
dipping the Si pyramid substrate into solution of hydrofluoric acid (HF) and 0.1 N silver nitrate (AgNO3) through a predetermined time to deposit Ag nanoclusters on surfaces of Si pyramid substrate;
etching the pyramid substrate in a solution of HF and Fe(NO3)3.9H2O to produce the so-called NWs/MPs substrate; and
removing residual Ag nanoclusters on the surface of the pyramid substrate by the ultrasonic vibration.

11. The method for fabricating NWs/MPs substrate complex structure as claimed in claim 10, wherein the process of etching the Si pyramid substrate is performed through 10 to 40 minutes so as to acquire nanowires lengths which are between 1.5 μm to 6.9 μm.

12. The method for fabricating NWs/MPs substrate complex structure as claimed in claim 10, wherein in above process, volumes and weights of all components in the process can be increased or decreased with the same ratio; this variation will not affect the fabricating process; and furthermore each value has a variation of ±20%.

13. The method for fabricating NWs/MPs substrate complex structure as claimed in claim 10, further comprising steps of modifying anti-EpCAM antibodies on the NWs/MPs substrate, comprising the steps of:
a surface of the NWs/MPs substrate being first modified with 3-mercaptopropyl trimethoxysilane (MPTMS) in absolute ethanol by silane chemistry for a predetermined time;
0.25 mM N-maleimidobutyryloxy succinimide ester (GMBS) in DMSO solution being added as a coupling agent for a predetermined time,
followed by streptavidin (SA) for a predetermined time; removing excess SA by using PBS (Phosphate buffered saline);
the modified NWs/MPs substrate being dipped into biotinylated antinylated anti-EpCAM antibodies through a predetermined time, and then the anti-EpCAM antibodies will connect with the SA on the nanowire 30.

14. The method for fabricating NWs/MPs substrate complex structure as claimed in claim 13, wherein in above process, volumes and weights of all components in the process can be increased or decreased with the same ratio; this variation will not affect the fabricating process; and furthermore each value has a variation of ±20%.

15. The method for fabricating NWs/MPs substrate complex structure as claimed in claim 10, further comprising the steps of:
sinking the NWs/MPs substrate into 0.002 M AgNO3 buffer solution through a predetermined time so as to form NWs/MPs substrate with Ag nanoparticles (AgNPs) on the surface, therefore the NWs/MPs substrate becomes a substrate with Surface enhanced Raman scattering (SERS); wherein volumes and weights of all components in can be increased or decreased with the same ratio; this variation will not affect the fabricating process; and furthermore each value has a variation of ±20%.

16. The method for fabricating NWs/MPs substrate complex structure as claimed in claim 15, wherein the AgNPs are bound with EBV probe DNAs; which can hybridize with EBV target DNAs so as to detect the concentration of EBV target DNAs in blood; above detecting method comprises the steps of:
the AgNPs are modified with 4-MBAs (4-Mercaptobenzoic acid) and EBV target DNAs; which comprises the steps of:
(1) adding EBV target DNAs and AgNPs to citrate buffer; then performing DNA loading at room temperature to form DNA-AgNPs mixture, then adding HEPES (4-(2-hydroxyethyl)-1-piperazineethanesulfonic acid) buffer to adjust the pH of the DNA-AgNPs mixture to neutral pH; then the DNA-AgNPs mixture being centrifuged, and then the supernatant EBV target DNAs being removed, and
then the DNA-AgNPs mixture being re-dispersed in HEPES buffer for further use;
(2) DNA-AgNPs mixture which contains AgNPs modified with the EBV target DNA being mixed with 4-MBA solution to form 4-MBA functionalized EBV target DNAs-AgNPs; the supernatant 4-MBA being removed by centrifuging; then the 4-MBA functionalized EBV target DNAs-AgNPs being dispersed in 1×PBS;
wherein the step of hybridization of EBV target DNAs bound on AgNPs with EBV probe DNA on substrate comprising the steps of:
(1) the EBV probe DNA being added on the AgNPs-coated NWs/MPs substrate which are incubated for a predetermined time at room temperature; and then they are washed with 1×PBS to remove unbound EBV probe DNAs, and
(2) then the DNA solution with EBV target DNA is added to the NWs/MPs substrate modified with EBV probe DNA for hybridization of target EBV DNA and probe DNA, and then Roman spectroscope is used for detection of hybridization of target EBV DNA and probe DNA.

17. The method for fabricating NWs/MPs substrate complex structure as claimed in claim 16, wherein a pH of citrate buffer solution is between 1 to 7; and a pH of HEPES is between 6 to 8.

18. The method for fabricating NWs/MPs substrate complex structure as claimed in claim 16, wherein pH of citrate buffer solution is 3 so as to have a preferred DNA adding effect; and the pH of HEPES is 7.6.

19. The method for fabricating NWs/MPs substrate complex structure as claimed in claim 16, wherein in above process, the volumes and weights of all components in the process can be increased or decreased with the same ratio; this variation will not affect the fabricating process of the present invention; and furthermore each value may have a variation of ±20%.

20. The method for fabricating NWs/MPs substrate complex structure as claimed in claim 10, wherein the material of the substrate is selected from carbon, silicon, or germanium.
